(19) United States
(12) Patent Application Publication
(10) Pub. No.: US 2012/0232481 A1
(43) Pub. Date: Sep. 13, 2012

(54) Retractable Syringe Needle Assembly

(76) Inventor: Richard Caizza, Vernon, NJ (US)

(21) Appl. No.: 13/413,567

(22) Filed: Mar. 6, 2012

Related U.S. Application Data
(60) Provisional application No. 61/450,007, filed on Mar. 7, 2011.

(51) Int. Cl. A61M 5/34; A61M 5/50

(52) U.S. Cl. 604/110; 604/240; 604/241

(57) ABSTRACT

A retractable needle syringe, including a needle stem and breakable ring having a plurality of fingers and grooves. These fingers and grooves are configured to mate with a plurality of ribs and grooves in a syringe barrel distal end to provide anti-rotation of the needle stem with respect to a syringe barrel. This anti-rotation feature restricts rotation of the needle stem when attaching or exchanging needles to prevent unintended retraction.

### CROSS REFERENCE TO RELATED APPLICATIONS

This application claims priority of U.S. Provisional Patent Application Ser. No. 61/450,007 entitled Retractable Syringe Needle Assembly, filed Mar. 7, 2011, the teachings of which are incorporated herein.

### BACKGROUND OF THE INVENTION

The present invention is directed to safety syringes, and particularly syringes with retractable needles configured to retract after use and prevent re-use and unintended sticking. Retractable syringes are known in the art, which typically include a needle configured to automatically retract into a syringe barrel after delivery of a medicant into a patient. The syringes typically have a fixed needle, meaning different sized needles cannot be selectively interchanged with a syringe barrel. These standard fixed needle arrangements are configured to aspirate fluids from a vial, which process can inadvertently damage the needle point while attempting to insert the needle into the vial, or during the handling process. Damaged needle points cause pain when inserted and withdrawn from patients and are undesirable. The need to have the ability to fill with a large bore needle particularly with viscous fluids and inject a patient with a small bore needle is preferred for patient comfort.

There is desired a retractable syringe having an interchangeable needle, configured such that the needle assembly can be securely attached to prevent fluid leakage and easily detached from the syringe.

### BRIEF DESCRIPTION OF THE FIGURES

FIG. 1 shows an exploded view of a retractable syringe having a Luer tip, as well as an interchangeable needle and hub forming a needle assembly configured to extend through the Luer Tip;

FIG. 2 shows a longitudinal cross sectional view of the syringe of FIG. 1 depicting a needle stem positioned in the syringe distal end and configured to receive the interchangeable needle assembly disposed through the Luer tip;

FIG. 3 shows an enlarged sectional view of the syringe distal end illustrating the needle stem having a threaded distal end configured to receive needles of various sizes through the Luer tip;

FIG. 4 shows a longitudinal cross sectional view of the syringe of FIG. 1 depicting the needle assembly after retraction into the syringe barrel;

FIG. 5 shows a cross section of the syringe barrel just distal of the segmented retaining ledge shown in FIG. 3.

FIG. 6 shows a perspective view of a retaining ring having fingers and recesses; and

FIG. 7 shows a partial sectional view of the barrel distal end including longitudinal ribs and grooves configured to engage with the retaining ring fingers and recesses.

### SUMMARY OF THE INVENTION

The present invention achieves technical advantages as a retractable needle syringe, including a needle stem and breakable ring having a plurality of fingers and grooves. These fingers and grooves are configured to mate with a plurality of ribs and grooves in a syringe barrel distal end to provide anti-rotation of the needle stem with respect to a syringe barrel. This anti-rotation feature restricts rotation of the needle stem when attaching or exchanging needles to prevent unintended rotational movement preventing the needle hub from torqueing on or off the needle stem.

### DETAILED DESCRIPTION OF A PREFERRED EMBODIMENT

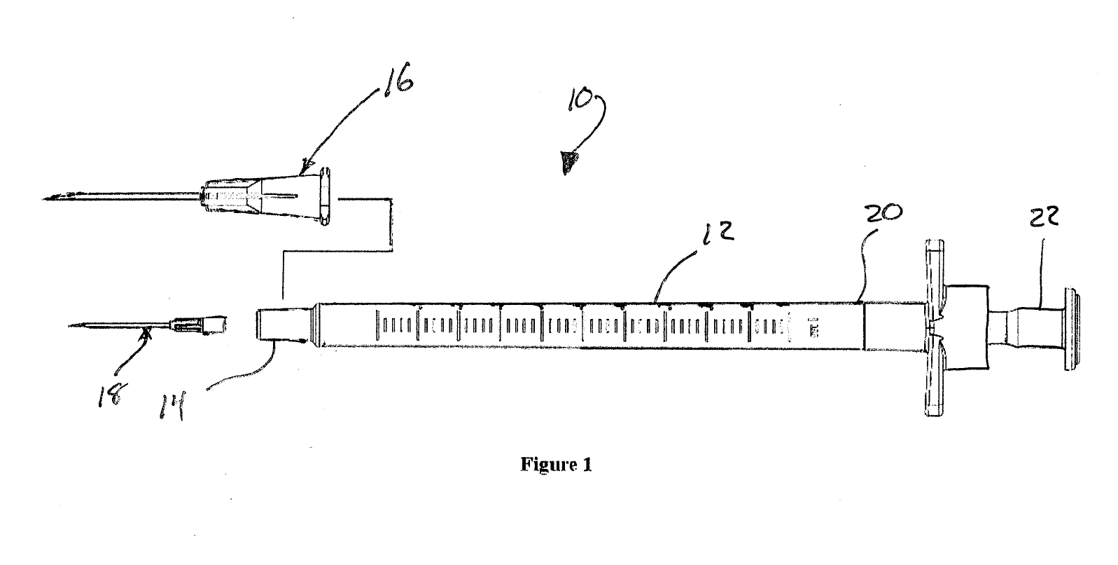

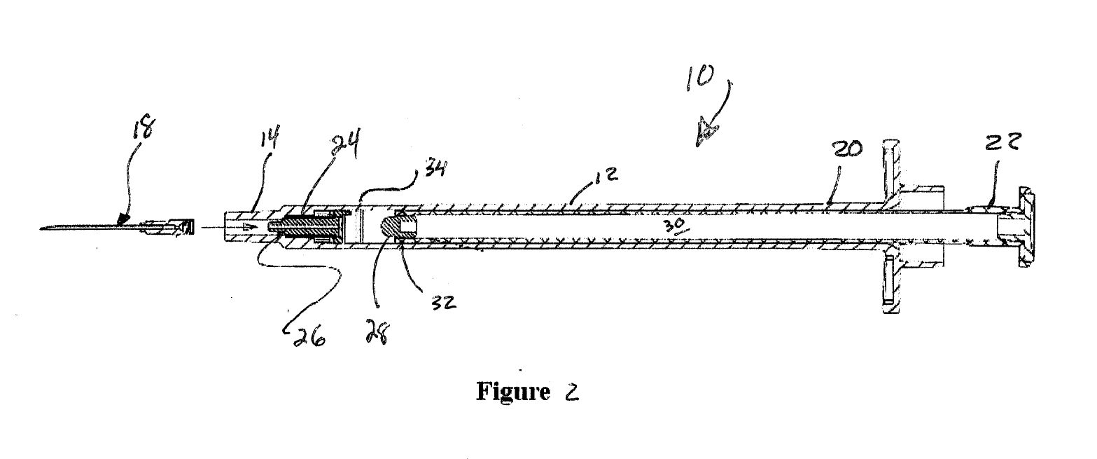

Referring to FIG. 1 there is shown a retractable needle syringe 10 having a syringe barrel 12 with a distal end 14 configured as a conical frustum tip. The conical frustum tip 14 is configured to attach to female Luer compatible devices including a filling needle & Luer hub 16 as shown, collectively referred to as a luer filling needle, as well as delivery tubes and the like. The syringe 10 is also shown to include a selectively attachable, interchangeable needle assembly 18 including a needle and threaded needle hub, allowing needles of different sizes and lengths to be interchanged with the syringe 10. Needle assembly 18 has radially extending ribs and is configured to be threadably coupled to a threaded needle stem 24 within the distal end of the conical frustum tip 14, as shown in the FIG. 2 as will be described shortly. Syringe 10 also includes a syringe proximal end 20 and a plunger 22 slidable therein from the proximal end, the plunger 22 configured to both aspirate a fluids through the Luer filling needle/hub 16, and also dispense the medicant upon compression. The plunger 22 is also configured to aspirate a fluid through the needle assembly 18 if desired. The filling needle/hub 16 may be desired as it is a common inexpensive needle that can also speed up the drawing process, and also prevents the possible unintentional retraction of the needle assembly 18 during insertion into the medicant vial or during the handling of the syringe 10 when drawing the medicant. Moreover, the conical frustum tip 14 advantageously allows the syringe 10 to be conveniently prefilled with medicant at one place and capped, then transported to a patient with or without needle assembly 18 as desired. This design is a significant advantage for many healthcare providers involved in the processing and handling of syringes until ultimate delivery of the medicant to a patient.

Referring to FIG. 2, there is shown a longitudinal cross sectional view of syringe 10 of FIG. 1, detailing the needle stem 24 having a threaded distal end 26 configured to receive the needle assembly 18. Notably, the needle stem 24 is positioned within the distal end of the conical frustum tip 14 and is advantageously protected from axial forces which could inadvertently being contacted and creating an unintended retraction of spring biased needle stem 24, such as when the needle assembly is secured to needle stem. Also shown is the plunger 22 having a plug 28 at a distal end thereof, which plug is in sealing arrangement with a cavity 30 of plunger 22 prior to retraction of needle assembly 18 therein, and which plug is dislodged into the cavity by the needle stem 24 that retracts with needle assembly 18 after an injection. The distal end of plunger 22 proximate the plug 28 has an integral seal 32 extending annularly thereabout. The interior surface of the syringe includes a plurality of annular detents 34 configured for positioning the seal 32 in a rested position, before the distal end of plunger 22 axially engages a protrusion 38 of an annular ring 36 coupled to the needle stem 24 by a breakable membrane, as more fully described in Applicant's U.S. Pat. No. 7,803,132 B1, the teaching of which are included herein by reference.

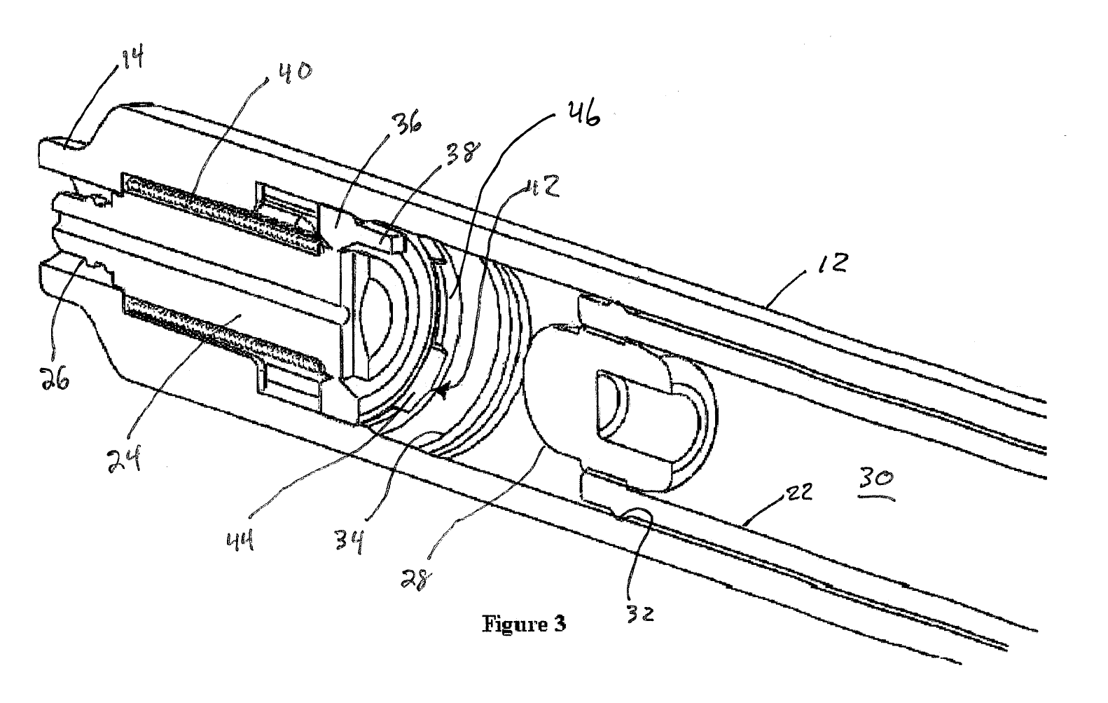

FIG. 3 depicts an enlarged cross sectional view of the needle stem 24, including the threaded needle distal end 26, positioned within the conical frustum tip 14, and coupled to the retaining ring 36 with protrusion 38 by the breakable membrane. Also shown is a spring 40 configured to retract the needle assembly 18 in response to the plunger 22 engaging the protrusion 38 after delivering the medicant to create a progressive separation of the retaining ring 36 from the needle stem 24 and the retraction of needle stem 24 into the plunger cavity 30. The annular detents 34 configured to seat position seal 32 are also shown.

This FIG. 3 also shows the interior surface of the barrel 12 including an annular retaining ledge generally shown at 42 configured to retain the needle stem 24 distally thereof such that the needle stem 24 is securely seated in the syringe barrel distal end, including during attachment of needle assembly 18, and until a complete retraction of the needle stem 24 and needle assembly 18 into cavity 30. Notably, the retaining ledge 42 is segmented, and is defined by segments of raised protrusions 44 extending from the interior barrel wall with notches or detents 46 defined between the protrusions 44 to provide several features. The segmented retaining ledge 42 enables reduced activation force required by the plunger to separate and break the retaining ring 36 from the needle stem 24, which provides an improved tactile feeling of the seal 32 and comfort of use. The segmented ledge 42 also makes it easier to install the needle stem 24 and retaining ring 36 in the barrel distal end, such that it snaps into place therepast with less axial force.

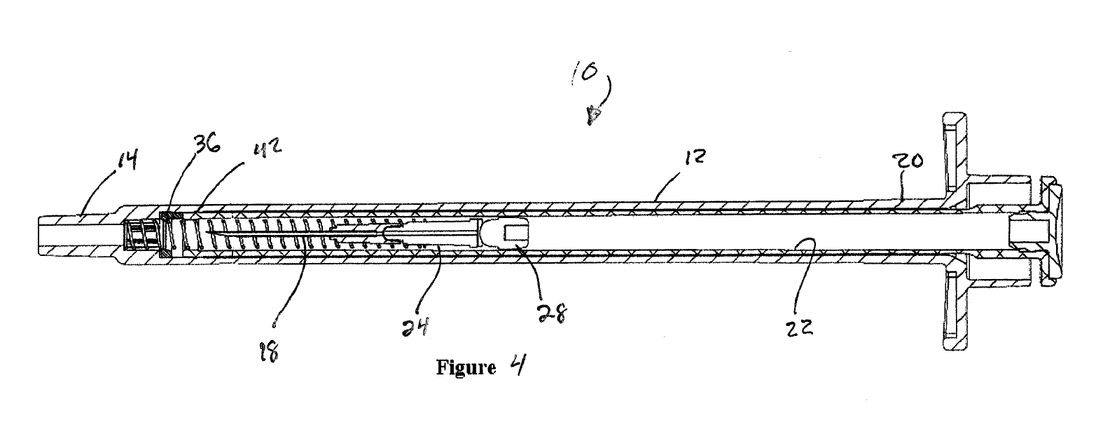

FIG. 4 depicts the needle stem 24 and needle assembly 18 after retraction into the plunger cavity 30.

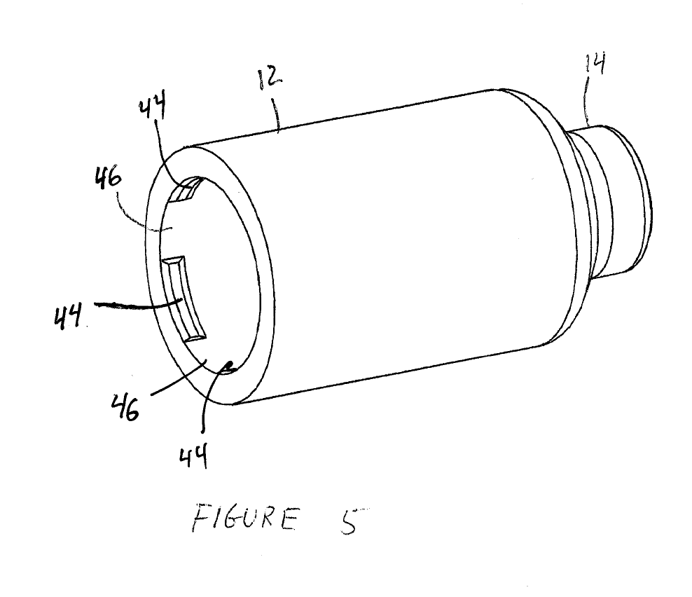

FIG. 5 depicts a cross section of the syringe barrel distal end taken proximate the segmented retaining ledge 42.

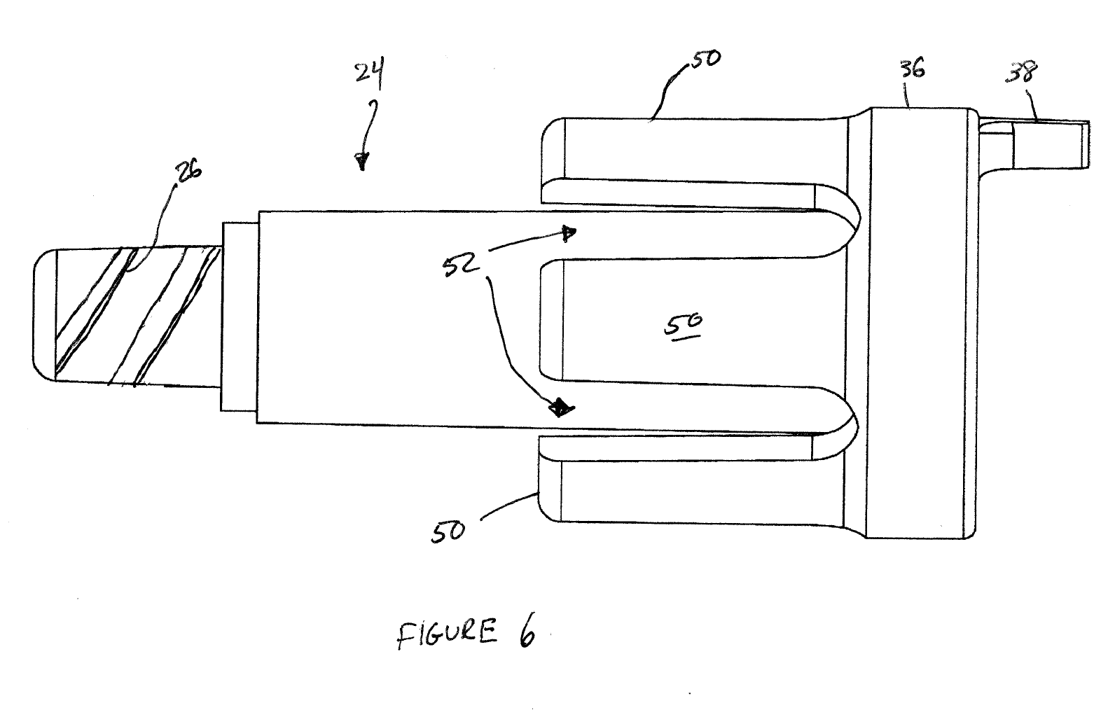

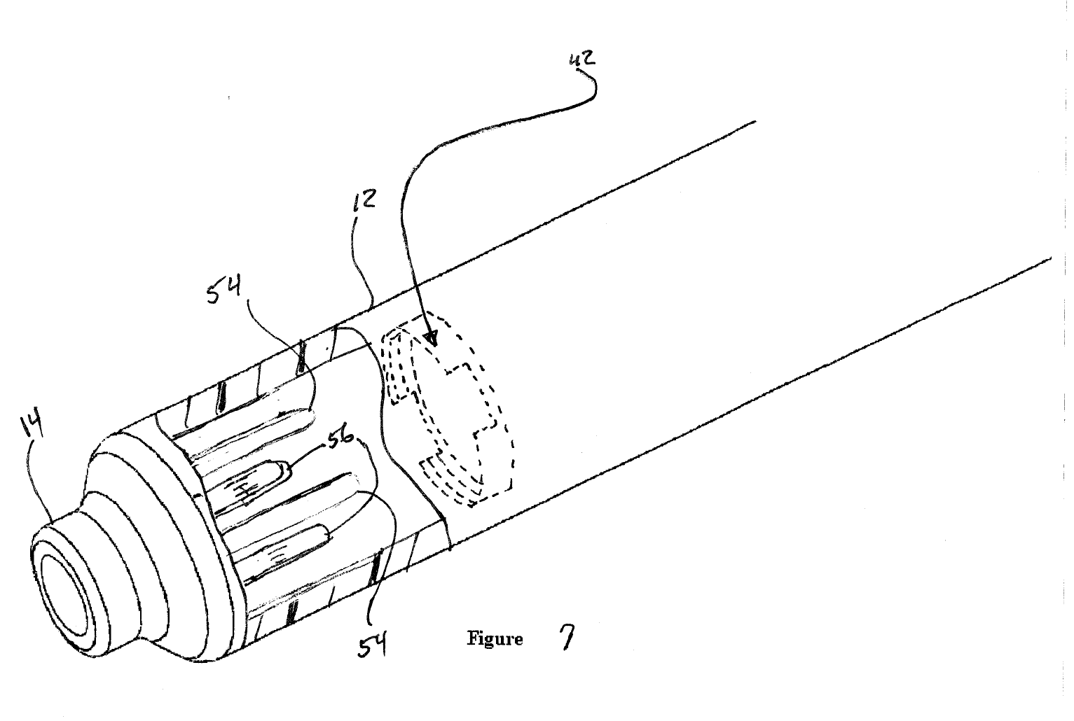

FIG. 6 depicts a perspective view of ring 36 including a plurality of longitudinally extending fingers 50 separated by longitudinally extending slots or recesses 52. The fingers and recesses extend distally of the retaining ring 36, and are uniformly spaced from each other and parallel to the exterior surface of needle stem 24, as shown. The fingers 50 have rounded exterior surfaces of a single diameter that seat in, and mate with, respective rounded, proximally extending and uniformly spaced grooves 54 defined in the interior wall of the barrel distal end, as shown in FIG. 7. The interior surface of the barrel distal end also has a plurality of uniformly spaced longitudinally extending ribs or splines 56 extending proximal from the barrel distal end, each side of the proximally extending grooves 54. The ribs 56 are configured to securely seat in the recesses 52 of ring 36 when the ring 36 and needle stem 24 are securely seated in the barrel distal end. The alternating fingers 50 and recesses 52 are configured to secure the ring 36 in place in the barrel distal end after assembly, and also provide anti-rotation such that the needle stem 24 cannot rotate, particularly when needle assemblies 18 are connected or exchanged with the threaded needle stem 26, or when a Luer device is attached to the Luer tip 14. This anti-rotation feature prevents the rotation of the needle stem 24 during attachment of the needle assembly 18 or other features. Any rotation of needle stem 24 could undesirably create leaking.

While the invention has been described in detail and with reference to a specific embodiment thereof, it will be apparent to one skilled in the art that various changes and modifications can be made therein without departing from the spirit and scope thereof. Thus, it is intended that the present invention covers the modifications and variations of this invention provided they come within the scope of the appended claims and their equivalents.

I claim:

1. A syringe, comprising:
a barrel having a proximal end and a distal end, and having a cavity configured to hold a fluid;
a plunger disposed in the barrel and configured to advance toward the barrel distal and proximal ends; and
a ring and needle stem disposed in and coupled to the barrel distal end, the needle stem configured to selectively couple to a needle assembly, the ring comprised of a plurality of longitudinally extending fingers configured to secure within the barrel distal end and configured to limit rotation of the needle stem with respect to the barrel during attachment and detachment of the needle assembly to the needle stem.

2. The syringe as specified in claim 1 wherein the ring further comprising a recess extending between each finger.

3. The syringe as specified in claim 2 wherein the barrel distal end has an inner surface comprising of at least one longitudinally extending member configured to extend in one of the recesses.

4. The syringe as specified in claim 3 wherein the longitudinally extending members are configured to limit rotational direction of the ring and needle stem in the barrel at the distal end after assembly therein.

5. The syringe as specified in claim 3 wherein the at least one longitudinally extending member comprises of at least one rib.

6. The syringe as specified in claim 5 wherein the at least one rib and the fingers limit rotational movement between the barrel and ring while allowing axial movement of the ring and needle stem in a distal direction.

7. The syringe as specified in claim 5 wherein the at least one rib and fingers allow a transmission of torque in one direction to create a liquid seal arrangement between the needle assembly and the needle stem.

8. The syringe as specified in claim 7 wherein the at least one rib and fingers allow a transmission of torque to allow removal of the needle assembly from the needle stem.

9. The syringe as specified in claim 5 wherein the at least one rib has a rounded exterior surface.

10. The syringe as specified in claim 2 wherein the barrel distal end has an inner surface comprising a plurality of longitudinally extending grooves configured to receive the fingers.

11. The syringe as specified in claim 3 wherein the barrel distal end inner surface comprises a plurality of longitudinally extending grooves configured to receive the fingers.

12. The syringe as specified in claim 1 wherein the needle stem is threaded and configured to rotatably receive the needle assembly.

13. The syringe as specified in claim 1 wherein the needle stem is spring biased toward the barrel proximal end.

14. The syringe as specified in claim 13 wherein the needle stem is configured to be separated from the ring by the plunger to permit retraction of the needle stem into the barrel upon delivery of the medicant.

15. The syringe as specified in claim 1 wherein the barrel distal end further comprises a Luer tip configured to couple to a Luer fill needle or likes to permit the syringe to draw the medicant there through, the Luer tip further configured to permit the needle assembly to be interchangeable with the needle stem.

16. The syringe as specified in claim 15 wherein the Luer tip is further configured to prevent unintended retraction of the needle stem into the barrel when the Luer tip is coupled to a Luer fill needle.

17. The syringe as specified in claim 16 wherein the needle stem is positioned closely proximate the Luer tip.

18. The syringe as specified in claim 17 wherein the needle stem is positioned within the Luer tip.

19. The syringe as specified in claim 15 wherein the Luer tip is configured to permit the needle assembly to axially extend there through.

20. The syringe as specified in claim 1 wherein the syringe is configured to prevent touch contamination of a portion of the needle assembly coupled to the needle stem .
